(19) United States
(12) Patent Application Publication
(10) Pub. No.: US 2005/0115872 A1
(43) Pub. Date: Jun. 2, 2005

(54) Catalyst for hydrorefining and/or hydroconversion comprising a novel active phase in the form of sulphide solid solution

(76) Inventors: Cecile Thomazeau, Rueil Malmaison (FR); Virginie Harle, Senlis (FR); Tivador Cseri, Courbevoie (FR); Michel Lacroix, Lyon (FR); Michel Danot, Nantes (FR); Christophe Geantet, Miribel (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/944,428

(22) Filed: Sep. 20, 2004

(30) Foreign Application Priority Data
Sep. 19, 2003 (FR) 03/11.032

(51) Int. Cl. C10G 45/06; B01J 27/02; B01J 27/043; C10G 45/60; C10G 47/02; C07C 5/32; C07C 4/12; C07C 5/02; C10G 45/48; B01J 27/045; C10G 45/04

(52) U.S. Cl. 208/216 R; 502/216; 502/222; 502/223; 502/325; 502/337; 502/338; 208/217; 208/111.3; 208/111.35; 208/251 H; 208/254 H; 208/143; 585/266; 585/275; 585/671; 585/670; 585/750; 585/661; 585/486

(57) ABSTRACT

The present invention relates to a catalyst for the hydrorefining and/or hydroconversion of hydrocarbon charges the active phase of which comprises at least one molybdenum and tungsten sulphide solid solution within the same flake, of approximate general formula MoₓW₁₋ₓS_{y}, where x is a number strictly comprised between 0 and 1 and y is a number comprised between 1.4 and 2.6 and preferably at least one element of Group VIII.

The present invention relates to a catalyst for the hydrorefining and/or hydroconversion of hydrocarbon charges, said catalyst comprising at least one mixed molybdenum and tungsten sulphide (Group VIB) within the same flake in the form of solid solution of formula MoₓW₁₋ₓ, S_{y}, optionally combined with a support comprising a porous matrix generally of the amorphous or crystallized oxide type, optionally combined with at least one metal of Group VIII of the periodic table of the elements and optionally combined with at least one doping element chosen from boron, phosphorus, silicon and the halogens.

The present invention also relates to the use of said catalyst for the hydrorefining and/or hydrocracking/hydroconversion of hydrocarbon charges such as the petroleum cuts, the cuts originating from carbon or the hydrocarbons produced from natural gas and more particularly from hydrocarbon charges containing heteroatoms.

Hydrorefining includes hydrogenation, hydrodenitrification, hydrodeoxygenation, hydrodearomatization, hydrodesulphuration, hydrodemetallization, hydroisomerization, hydrodealkylation, dehydrogenation reactions.

The hydrorefining of the hydrocarbon charges such as the sulphurated petroleum cuts is becoming more and more important within the practice of refining with the growing necessity to reduce the quantity of sulphur present in the petroleum products and to convert heavy fractions to lighter fractions which can be used as fuels. This state of affairs is due on the one hand to the economic advantage of making the best use of imported crudes which are increasingly rich in heavy fractions, poor in hydrogen and rich in heteroatoms, including nitrogen and sulphur, and on the other hand to the specifications imposed on commercial fuels in various countries. This valorization involves a relatively great reduction in the molar mass of the heavy constituents, which can be obtained for example by means of cracking and hydrocracking reactions of charges which have been previously refined, i.e. desulphurated and denitrified.

Current catalytic hydrorefining processes use catalysts capable of promoting the main reactions required for utilizing heavy cuts, in particular hydrogenation of the aromatic rings (HAR), hydrodesulphuration (HDS), hydrodenitrification (HDN) and other hydroelimination processes. Hydrorefining is used for treating charges such as gasolines, gasoils, vacuum gasoils, atmospheric or vacuum residues, deasphalted or not deasphalted. For example, it is completely indicated for the pre-treatment of the charges for catalytic cracking and hydrocracking processes. At least one hydrorefining stage is usually integrated into each of the known schemes for the valorization of the petroleum cuts.

The context of the present invention, summarized below, is well known to a person skilled in the art.

The problem posed to a person skilled in the art is the obtaining of high catalytic performances: activity and selectivity, with production envisageable on an industrial scale.

We have found that, preferably in combination with a promoter of Group VIII, the ternary system MoₓW₁₋ₓS_{y} leads to catalytic activities which are distinctly greater than those of the mechanical mixture of the sulphides MoSu_{y} and WS_{y} taken in the same proportions. EXAFS characterization of these systems has made it possible to detect the presence of molybdenum and tungsten within the same flake, i.e. the formation of a sulphide solid solution, in general of lamellar morphology. It would seem that the particular properties of this active phase at the origin of this increase in catalytic activity result from the formation of this solid solution.

### DESCRIPTION OF THE INVENTION

The invention thus relates to a catalyst for the hydrorefining and/or hydroconversion of hydrocarbon charges in which the active phase comprises at least one molybdenum and tungsten sulphide solid solution. Said hydrorefining and/or hydroconversion catalyst optionally comprises at least one element of Group VIII, preferably chosen from the group formed by iron, cobalt, nickel. The catalyst optionally contains a matrix, generally amorphous, such as alumina or a silica-alumina. The catalyst optionally contains a zeolite, such as for example a Y zeolite with a faujasite structure. The catalyst also optionally contains at least one doping element chosen from boron, phosphorus, silicon and the halogens.

Said hydrorefining and/or hydroconversion catalyst has an activity of hydrogenation of the aromatic hydrocarbons and of hydrodesulphuration which is greater than that of the catalytic formulae known in the prior art. Without wanting to be bound by any theory, it seems that this particularly high level of activity of the catalysts according to the invention is due to the particular properties of the sulphide solid solution of the two elements of Group VIB (molybdenum and tungsten) allowing an improvement in the hydrogenating and hydrodesulphurizing properties. The synergy effect of this sulphide solid solution makes it possible, with the catalyst according to the invention, to obtain catalytic activities greater than the weighted average of the catalytic activities which would be obtained by each of the single sulphides taken separately.

The solid solution present in the catalyst of the present invention is characterized by the following approximate general formula: MoₓW₁₋ₓS_{y} where:

x is a number strictly comprised between 0 and 1, preferably comprised between 0.005 and 0.995 and y is a number close to 2, generally comprised between 1.4 and 2.6. Structures similar to the lamellar structure of the disulphide are generally obtained.

The catalyst according to the invention can be in supported form, i.e. it comprises a support containing at least one matrix, or in mass form, i.e. it does not comprise any matrix.

The mass catalyst according to the present invention generally contains in wt. % relative to the total mass of the catalyst:
0.01 to 100%, preferably 0.1 to 100% and still more preferably 1 to 100% of at least one mixed molybdenum and tungsten sulphide phase forming a solid solution, the catalyst being able to contain moreover 0 to 99.95%, preferably 0 to 99.9% and still more preferably 0 to 99% of at least one element of Group VIII.

The supported catalyst according to the present invention generally contains, in wt. % relative to the total mass of the catalyst:
1 to 99.9%, preferably 5 to 99.5% and still more preferably 10 to 99% of at least one matrix,
0.1 to 99%, preferably 5 to 95% and still more preferably 1 to 90% of at least one mixed molybdenum and tungsten sulphide phase forming a solid solution, in particular the disulphide, the catalyst being able to contain moreover 0 to 30%, preferably 0 to 25% and still more preferably 0 to 20% of at least one metal of Group VIII, 0 to 90%, preferably 0 to 80%, more preferably 0 to 70% and still more preferably 0 to 60% of at least one zeolite molecular sieve, for example a Y zeolite with a faujasite structure, generally in hydrogen form.

The mass or supported catalyst can optionally comprise 0 to 20%, preferably 0.1 to 15% and still more preferably 0.1 to 10% of at least one doping element chosen from the group formed by phosphorus, boron, silicon and the halogens such as chlorine and fluorine.

Detection of the Solid Solution

Principle of EXAFS

The solid solution of molybdenum-tungsten sulphide is characterized by the EXAFS (Extended X-Ray Absorption Fine Structure, or X-ray absorption spectroscopy) technique. This technique makes it possible to obtain structural information on the local environment of a given atom, whatever the physical state of the material (crystalline or amorphous solid, liquid, gas). The X photons used during this type of characterization are high-energy and high-brilliance X photons produced by a synchrotron source. The synchrotron radiation is obtained from relativistic electrons or positrons subjected to the centripetal acceleration of a magnetic field. These relativistic positrons emit rays (X photons) along the tangent of their circular trajectory.

At the macroscopic level, when the X rays pass through the material, the intensity I of the beam transmitted after passing through a homogeneous sample, of thickness x and absorption coefficient μ, is lower than the intensity lo of the incident beam. The intensity ratio I/Io obeys Beer-Lambert's Law I/Io=e^{-μx}.

At the microscopic level, the photon of energy hν excites an electron in the internal layers of the absorbing atom (1 s at the K threshold, 2 p at the L_{III} threshold) which is ejected in the form of a photoelectron if the energy hν of the incident photon is greater than the ionization energy of the absorbing atom. If the photon energy is lower than the ionization energy of the absorbing atom, it is the linked excited states corresponding to the XANES (X-ray Absorption Near Edge Structure) which are explored. The photoelectron ejected is propagated in the medium with a kinetic energy and wave associated with a wave vector k. In the case of a non-isolated atom, there is interference between the leaving wave and the wave back scattered by the neighbouring atoms. The interference with the leaving wave periodically modulates the absorption coefficient. Oscillations are obtained, they correspond to the EXAFS signal. The processing of these oscillations makes it possible to extract a Fourier transform, which corresponds to the radial distribution of the atoms around the absorbing atom.

Modelling and Identification of the Solid Solution

X-ray absorption makes it possible to determine the composition (molybdenum and/or tungsten) of the flakes for these non-crystalline materials. The characterizations are carried out at the K threshold of the molybdenum in transmission, and L_{III} threshold of the tungsten in transmission or in fluorescence according to the contents of absorbing element in the catalyst.

At present EXAFS is the only appropriate technique for differentiating between molybdenum and tungsten in the same flake. In fact, the molybdenum and tungsten sulphides have very similar structural characteristics (mesh parameters). Thus, the substitution of tungsten for molybdenum does not lead to any measurable variation in the inter-atomic distances. It is therefore the variation in the electronic characteristics (amplitude of back scatter and phase shift induced by the back scattered atom) which makes it possible to decide on the existence of a solid solution on the basis of the X-ray absorption spectra. The identification of the solid solution of molybdenum-tungsten sulphide is then carried out by modelling the absorption spectra obtained by EXAFS by introducing these electronic parameters into a theoretical model assuming the existence of a solid solution. If the EXAFS signal is adjusted to the theoretical model, the presence of a solid solution is detected.

The EXAFS signal is formed of oscillations which make it possible to characterize the local environment of an absorbing atom (inter-atomic distances, number and nature of neighbours). In the case of a solid solution of molybdenum-tungsten sulphide, since the inter-atomic distances cannot supply any information, the parameters to be identified by modelling the EXAFS signal are the nature and number of neighbours.

Analysis of the X-ray absorption spectra makes it possible to visualize the radial distribution function of the atoms around the absorbing element by extraction of the Fourier transform from the EXAFS signal. This radial distribution has characteristic peaks. The x-axis of these peaks is linked to the position of the atoms around the absorbing atom. The intensity of these peaks is linked to the number of atoms situated at the distance given by the x-axis of the peaks considered. The description below is given for a disulphide system, but it is applicable to any sulphide according to the invention.

For the disulphide MoS₃, the first peak of the Fourier transform corresponds to the sulphurous environment of the absorbing molybdenum atom (first coordination sphere), the second peak corresponds to the molybdenum neighbours situated in the same flake as the absorbing molybdenum (second coordination sphere). For the disulphide WS₂ the first peak of the Fourier transform corresponds to the sulphurous environment of the absorbing tungsten atom, the second peak corresponds to the tungsten neighbours situated in the same flake as the absorbing tungsten.

In the presence of a solid solution of molybdenum-tungsten sulphide, the radial distribution is modified qualitatively. The sulphurous environment around the absorbing atom (molybdenum or tungsten) remains identical, the first peak of the Fourier transform is not affected. The modifications to the radial distribution appear at the level of the second peak of the Fourier transform.

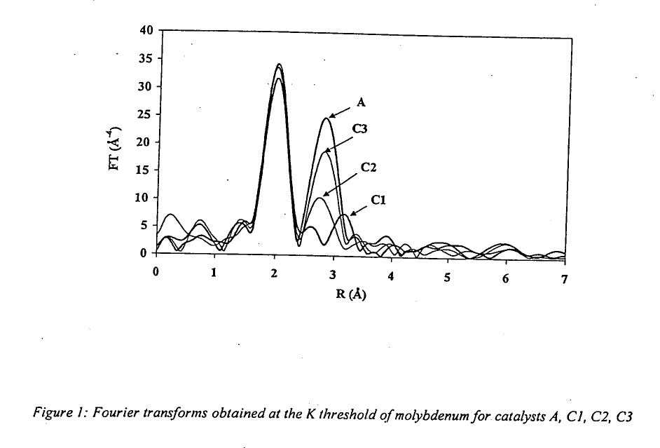

When the solid solution is characterized at the K threshold of the molybdenum, the intensity of this second peak diminishes considerably when the level of substitution of tungsten for molybdenum increases, i.e. when the tungsten content increases. This is characteristic of the presence of molybdenum and tungsten within the same flake. For sufficiently large tungsten contents, a third peak appears, characteristic of the tungsten contribution (FIG. 1, catalysts C3 to C2). This peak is masked by the molybdenum contribution for lower tungsten contents (FIG. 1, catalyst C₁).

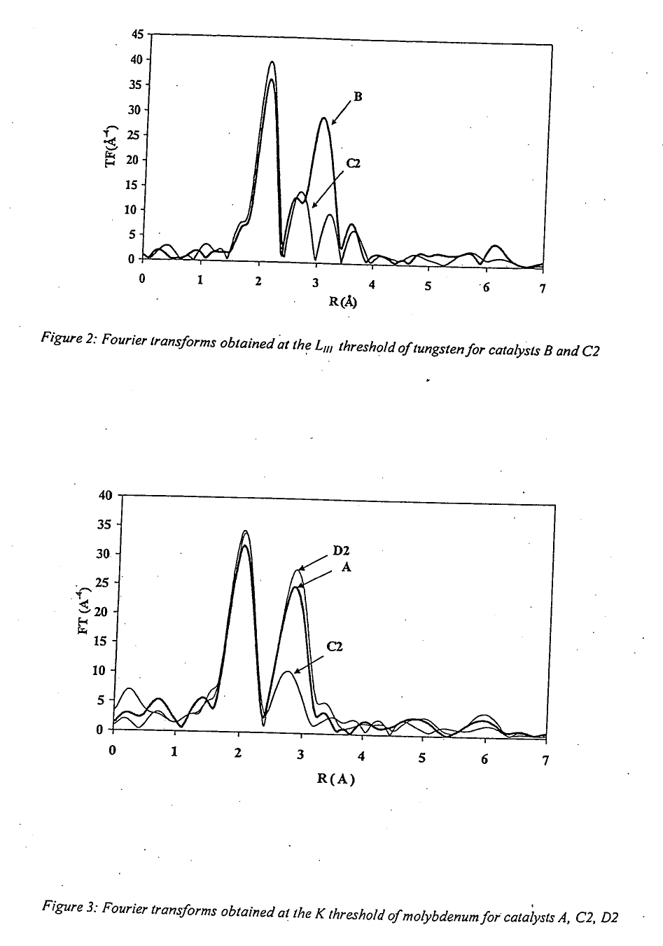

When the solid solution is characterized at the L_{III} threshold of tungsten, the second peak initially observed for the WS₂ sulphide (FIG. 2, catalyst B) is split into two distinct peaks, characteristic of the presence of molybdenum and tungsten within the same flake (FIG. 2, catalyst C2).

In the absence of a solid solution, the Fourier transforms of the mixed sulphides remain identical to the Fourier transform of molybdenum sulphide when the characterization is done at the K threshold of molybdenum and identical to the Fourier transform of tungsten sulphide when the characterization is done at the L_{III} threshold of tungsten (FIG. 3). This situation expresses the coexistence of MoS₂ flakes and WS₂ flakes (biphasing).

Preparation of the Solid Solution for the Active Sulphide Phase

Generally, the process for preparing the mass mixed sulphide comprised in the catalyst of the present invention includes the formation of a reaction mixture which contains at least the following compounds: at least one source of molybdenum, at least one source of tungsten, optionally water, optionally a non-aqueous solvent, optionally at least one element chosen from the group formed by the elements of Group VIII, optionally doping elements chosen from the group formed by boron, phosphorus, silicon and the halogens.

The mass catalysts of the present invention can be prepared by any appropriate methods. Preferably, the source of molybdenum is ammonium thiomolybdate, the source of tungsten is ammonium thiotungstate.

More particularly, the process for preparing the mass catalyst of the present invention comprises the following stages:
a) a proportion x of molybdenum precursor such as ammonium thiomolybdate and a proportion (1-x) of tungsten precursor such as ammonium thiotungstate, optionally an element of Group VIII precursor, and optionally a doping element precursor are dissolved in an appropriate volume of aqueous solution.
b) the solvent is gradually evaporated off, at a temperature comprised between 10 and 80° C.
c) the solid obtained is decomposed by an activation treatment by sulphuration.

The supported catalysts of the present invention can be prepared by any appropriate methods. Preferably, the support, for example a commercial alumina, is impregnated by an aqueous solution containing at least both molybdenum and tungsten (co-impregnation).

More particularly, the process for preparing the supported catalyst of the present invention comprises the following stages:
a) the support, for example a commercial alumina, is impregnated by an aqueous solution containing both molybdenum and tungsten and optionally an element of Group VIII, and optionally a doping element.
b) the damp solid is left to rest under a humid atmosphere at a temperature comprised between 10 and 80° C.
c) the solid obtained in Stage b) is dried at a temperature comprised between 60 and 150° C.
d) the solid obtained in Stage c) is calcined at a temperature comprised between 150 and 800° C.
e) activation of the catalyst is carried out by sulphuration.
   Impregnation of the Support

Impregnation of the support is preferably carried out by the so-called “dry” impregnation method well known to a person skilled in the art. The impregnation is very preferably carried out in a single stage by a solution containing all of the constitutive elements of the final catalyst (co-impregnation).

Other impregnation sequences can be implemented in order to obtain the catalyst of the present invention.

Thus, it is possible to carry out, in various orders, successive impregnations, each supplying one or more of the constitutive elements of the catalyst, each of these impregnations being followed by drying and calcination.

Another well-known method consists of introducing one or more of the constitutive elements during the synthesis of the support. The other elements can be introduced by impregnation of the solid thus obtained.

The sources of elements of Group VIB (Mo, W) which can be used are well known to a person skilled in the art.

For example, among the sources of molybdenum, it is possible to use molybdenum oxides and hydroxides, molybdic acid, phosphomolybdic acid, silicomolybdic acid, molybdenum acetylacetonate, molybdenum xanthate, ammonium dimolybdate, ammonium heptamolybdate, molybdenum salts such as molybdenum fluoride, molybdenum chloride, molybdenum oxybromide, molybdenum bromide, molybdenum iodide, molybdenum oxyfluoride, molybdenum oxychloride, molybdenum oxybromide, molybdenum oxyiodide, molybdenum hydride, molybdenum nitride, molybdenum oxynitride, molybdenum boride, molybdenum carbide, molybdenum oxycarbide, molybdenum phosphide, molybdenum sulphide, molybdenum hexacarbonyl, thiomolybdates, molybdenum thiophosphates, molybdenum xanthates and thioxanthates, molybdenum dithiophosphates, dithiocarbonates and dithiophosphinates, molybdenum carboxylates. Molybdenum oxide, ammonium dimolybdate, ammonium heptamolybdate or phosphomolybdic acid are preferably used.

For example, among the sources of tungsten, it is possible to use tungsten oxides and hydroxides, tungstic acid, ammonium metatungstate, phosphotungstic acid, silicotungstic acid, tungsten acetylacetonate, tungsten xanthate, tungsten fluoride, tungsten chloride, tungsten bromide, tungsten iodide, tungsten oxyfluoride, tungsten oxychloride, tungsten oxybromide, tungsten oxyiodide, carbonyl complexes, thiotungstates, tungsten thiophosphates, tungsten acetates, xanthates and thioxanthates, tungsten dithiophosphates, dithiocarbamates and dithiophosphinates, tungsten carboxylates. Tungsten oxide, ammonium tungstate, ammonium metatungstate or phosphotungstic acid are preferably used.

The doping precursors are chosen from the boron, silicon, phosphorus precursors and elements of the group of halogens. The preferred source of phosphorus is orthophosphoric acid H₃PO₄, but its salts and esters such as the alkaline phosphates, ammonium phosphates, gallium phosphates or alkyl phosphates are also suitable. The phosphorous acids, for example hypophosphorous acid, phosphomolybdic acid and its salts, phosphotungstic acid and its salts can be advantageously used. Phosphorus can for example be introduced in the form of a mixture of phosphoric acid and a basic organic compound containing nitrogen such as ammonium hydroxide, primary and secondary amines, cyclic amines, compounds of the family of pyridine and the quinolines, and compounds of the pyrrole family.

Numerous sources of silicon can be used. Thus it is possible to use ethyl orthosilicate Si(OEt)₄, siloxanes, halide silicates such as ammonium fluorosilicate (NH₄)₂SiF₆ or sodium fluorosilicate Na₂SiF₆. Silicomolybdic acid and its salts, silicotungstic acid and its salts can be advantageously used. Silicon can be added for example by impregnation of ethyl silicate in solution in a water-alcohol mixture. Silicon can be added for example by impregnation of a silicon compound having the following general formula: R₁(R₂R₃Si—O)xR₄ with R₁, R₂, R₃, R₄ being able to be individually one of the following groups: —R, OR, COOR, SiR₅R₆R₇, —Cl, —F, —Br, —I with R₅, R₆, R₇ being able to be individually chosen from one of the following groups: H, or an alkyl, aromatic, cycloalkane, alkylaromatic, alkylcycloalkane, naphthene, naphthenoaromatic radical, of formula CₙH_{2n-y}, with x=1 to 100, n=1 to 20, and y an odd integer comprised between −1 and 29 such that 2n-y is greater than zero.

The source of boron can be boric acid, preferably orthoboric acid H₃BO₃, ammonium biborate or pentaborate, boron oxide, boric esters of formulae B(OR)₃ and HB(OR)₂ in which R is a hydrocarbon radical usually having 1 to 50 carbon atoms and being able to comprise heteroatoms in the chain or as substituents on the chain. As examples of hydrocarbon radicals, methyl, ethyl, propyl, butyl, pentyl, pentyl, heptyl and octyl radicals can be mentioned. The R groups in the above formulae can be identical to or different from one another. Boron can be introduced for example by a solution of boric acid in a water-alcohol mixture.

The sources of elements of the group of halogens which can be used are well known to a person skilled in the art. For example, fluoride anions can be introduced in the form of hydrofluoric acid or its salts. These salts are formed with alkali metals, ammonium or an organic compound. In the latter case, the salt is advantageously formed in the reaction mixture by reaction between the organic compound and the hydrofluoric acid. It is also possible to use hydrolyzable compounds which can release fluoride anions into the water, such as ammonium fluorosilicate (NH₄)₂SiF₆, silicon tetrafluoride SiF₄ or sodium tetrafluoride Na₂SiF₆. Fluorine can be introduced for example by impregnation of an aqueous solution of hydrofluoric acid or ammonium fluoride.

The catalyst of the present invention can contain an element of Group VIII such as iron, cobalt, nickel, ruthenium, rhodium, palladium, osmium, iridium or platinum. Among the metals of Group VIII it is preferable to use a metal chosen from the group formed by iron, cobalt, nickel and ruthenium. Advantageously, combinations of the following metals are used: nickel-molybdenum-tungsten, cobalt-molybdenum-tungsten, iron-molybdenum-tungsten. It is also possible to use combinations of four metals, for example nickel-cobalt-molybdenum-tungsten. It is also possible to use combinations containing a noble metal such as ruthenium-molybdenum-tungsten, or also ruthenium-nickel-molybdenum-tungsten.

The sources of elements of Group VIII which can be used are well known to a person skilled in the art. For example, for non-noble metals, nitrates, sulphates, phosphates, halides, for example chlorides, bromides and fluorides, the carboxylates for example acetates and carbonates will be used. For the noble metals, halides, for example chlorides, nitrates, acids such as chloroplatinic acid, and oxychlorides such as ammoniacal ruthenium oxychloride will be used.

The supported catalyst of the present invention also contains at least one porous mineral matrix, usually amorphous or poorly crystallized. This matrix is usually chosen from the group formed by alumina, silica, silica-alumina, magnesia, clay, titanium oxide, zirconium oxide, lanthanum oxide, cerium oxide, aluminium phosphates, boron phosphates or a mixture of at least two of the abovementioned oxides and the combinations alumina boron-oxide, alumina titanium-oxide, alumina zirconia and titanium oxide-zirconia. It is also possible to choose aluminates, for example magnesium calcium, barium, manganese, iron, cobalt, nickel, copper and zinc aluminates, mixed aluminates and for example those containing at least two of the abovementioned metals. It is also possible to choose titanates, for example, zinc, nickel, cobalt titanates. It is preferable to use matrices containing alumina, in all its forms known to a person skilled in the art, for example gamma alumina.

Mixtures of alumina and silica and mixtures of alumina and boron oxide can be used advantageously.

The matrix can also contain, in addition to at least one of the abovementioned compounds, at least one synthetic or natural simple clay of the 2:1 dioctehedral phyllosilicate or 3:1 trioctahedral phyllosilicate type, such as kaolinite, antigorite, chrysotile, montmorillonite, beidellite, vermiculite, talc, hectorite, saponite, laponite. These clays can be optionally delaminated.

Mixtures of alumina and clay and mixtures of silica-alumina and clay can also be used advantageously.

The support can also contain, in addition to at least one of the abovementioned compounds, at least one compound chosen from the group formed by the family of molecular sieves of crystallized aluminosilicate type or synthetic or natural zeolites such as Y zeolite, fluorinated Y zeolite, Y zeolite containing rare earths, X zeolite, L zeolite, beta zeolite, mordenite with small pores, mordenite with large pores, omega zeolites, Nu-10, ZSM-22, Nu-86, Nu-87, Nu-88 and the ZSM-5 zeolite.

Among the zeolites, it is usually preferable to use the zeolites, the silicon/aluminium (Si/Al) atom ratio of the framework of which is greater than approximately 3:1. Zeolites with a faujasite structure are advantageously used and in particular stabilized Y and ultrastabilized USY zeolites either in a form at least partially exchanged with metallic cations, for example cations of the alkaline earth metals and/or cations of rare earth metals with an atomic number of 57 to 71 inclusive, or in hydrogen form (Zeolite Molecular Sieves Structure, Chemistry and Uses, D. W. BRECK, J. WILLEY and sons 1973).

The catalyst can also contain at least one compound chosen from the group formed by the family of non-zeolite crystallized molecular sieves such as the mesoporous silicas, silicalite, silicoaluminophosphates, aluminophosphates, ferrosilicates, titanium silicoaluminates, the borosilicates, chromosilicates and aluminophosphates of transition metals (including cobalt).

Sulphuration

The catalysts of the present invention are preferably subjected to a sulphuration treatment making it possible to convert, at least in part, the metallic species to sulphides before they are brought into contact with the charge to be treated.

The source of sulphur can be sulphur itself, carbon, hydrogen sulphide, sulphurous hydrocarbons such as dimethylsulphide, dimethyldisulphide, mercaptans, thiophene compounds, thiols, polysulphides such as for example ditertiononylpolysulphide or TPS-37 from the company ATOCHEM, petroleum cuts which are rich in sulphur such as gasoline, kerosene, gasoil, alone or in mixtures with one of the abovementioned sulphurous compounds. The preferred source of sulphur is hydrogen sulphide or dimethyl disulphide.

A standard method of sulphuration well known to a person skilled in the art consists of heating in the presence of hydrogen sulphide (pure or for example under a flow of a hydrogen/hydrogen sulphide) at a temperature comprised between 150 and 800° C., preferably between 250 and 600° C., generally in a fluidized bed reaction zone.

Preferably, the sulphuration is carried out ex situ, for example according to the TOTSUCAT sulphuration process described in U.S. Pat. No. 6,100,216 (FR 2 743 512). This process comprises the incorporation of a sulphurizing agent chosen for example from the group formed by elementary sulphur and the organic polysulphides into a catalyst and to a greater or lesser extent into the pores of the catalyst. The incorporation is carried out in the presence of a solvent which contains at least in part an olefmic compound or fraction, such as for example a vegetable oil or a similar component. The process comprises the treatment of the catalyst with hydrogen between 150 and 700° C., and then an oxidizing passivation stage. The sulphuration procedure is carried out at a site different from the site of final use, i.e. outside the reactor in which the catalyst will be used for its petroleum fractions conversion or hydroconversion function (ex-situ sulphuration).

The preferred sulphuration treatment consists of bringing the catalyst in contact ex-situ with hydrogen and H₂S or with hydrogen and a sulphurated compound which can be converted to H₂S in the presence of hydrogen at a temperature comprised between 250 and 600° C. The sulphuration reaction is thus carried out in gaseous phase.

More preferably, the temperature of the sulphuration stage is comprised between 350 and 600° C., still more preferably between 350 and 500° C.

The sulphuration can be carried out in one or two stages. In the one-stage embodiment, hydrogen sulphide or a sulphurated compound intended to be decomposed immediately into hydrogen sulphide is introduced in mixture or simultaneously with the hydrogen.

The two-stage embodiment comprises a first stage in which the catalytic compound is carefully mixed with elementary sulphur or a sulphurated compound other than hydrogen sulphide and in the absence of hydrogen, optionally in the presence of a solvent to which a vegetable or olefmic oil has optionally been added, and in which it is possible to operate in the presence of steam and/or inert gas. In the second stage, the catalytic compound into which the sulphur is incorporated is brought into contact with hydrogen optionally in the presence of steam.

At the end of the one or two-stage reaction, the catalyst is purged under inert gas in order to evacuate the hydrogen at least in part as well as the hydrogen sulphide at least in part from in its pores.

The sulphuration procedure can be carried out on a catalyst in motion in the sulphur-incorporation zone or in a fixed bed.

In one very preferred embodiment, the ex-situ sulphuration procedure of the catalysts according to the invention is carried out in gas phase at a temperature comprised between 350 and 600° C., under a flow of H₂/H₂S, the pressure being advantageously equal to atmospheric pressure.

Uses

The catalysts obtained by the present invention are used for hydrorefining and hydrocracking of hydrocarbon charges such as petroleum cuts, cuts originating from carbon or the hydrocarbons produced from natural gas and more particularly for hydrogenation, hydrodenitrification, hydrodeoxygenation, hydrodearomatization, hydrodesulphuration, hydrodemetallization hydroisomerization, hydrodealkylation, dehydrogenation and hydrocracking of hydrocarbon charges containing aromatic and/or olefinic, and/or naphthenic, and/or paraffinic compounds, said charges optionally containing metals, and/or nitrogen, and/or oxygen and/or sulphur. In these uses, the catalysts obtained by the present invention have an improved activity relative to the prior art.

Charges

The charges used in the process are generally chosen from the group formed by gasolines, gasoils, vacuum gasoils, deasphalted or non-deasphalted residues, vacuum residues, paraffinic oils, waxes and paraffins and effluents from the Fischer-Tropsch process. They contain at least one heteroatom such as sulphur, oxygen and optionally metals such as nickel and vanadium. The hydrorefining or hydrocracking conditions, such as temperature, pressure, litre of hydrogen/litre of hydrocarbon volume ratio, hourly space velocity, can be very variable as a function of the nature of the charge, the quality of the desired products and installations at the refiner's disposal. The operating conditions used in the reactor or reactors of the process according to the invention are generally: a temperature above 200° C., often comprised between 200 and 450° C., under a pressure often comprised between 1 and 30 MPa, preferably below 20 MPa, the space velocity being comprised between 0.1 and ₁₀^{h-1} and preferably 0.1-6^{h-1}, preferably 0.1-4^{h-1}, and the quantity of hydrogen introduced is such that the litre of hydrogen/litre of hydrocarbon volume ratio is comprised between 10 and 5000 l/l and most often between 10 and 2000 l/l.

### EXAMPLES

The following examples illustrate the invention without limiting its scope.

### Example 1

### Supported NiMo Catalyst (Comparative)

Molybdenum and nickel are co-impregnated dry in an aqueous medium on a commercial γ alumina support in the form of extrudates, developing a specific surface area of 250 m²/g. The molybdenum precursor salt is ammonium heptamolybdate (NH₄)₆Mo₇O₂₄, 4 H₂O, the nickel precursor salt is nickel nitrate Ni(NO₃)₂, 6 H₂O. The quantity of nickel is adjusted in order to keep to the molar ratio Ni/(Ni+Mo)=0.3.

After soaking for 6 hours, the extrudates are dried at 120° C. overnight, then calcined under air at 500° C. for 2 hours. On this non-sulphurated catalyst Ni—MoO₃/Al₂O₃, the molybdenum content is 8.3%, which corresponds to 0.104 mole of the element molybdenum per 100 g of catalyst, the nickel content is 2.0%, which corresponds to 0.045 mole of the element nickel per 100 g of catalyst. This catalyst A is representative of an industrial catalyst.

### Example 2

### Supported NiW Catalyst (Comparative)

Tungsten and nickel are co-impregnated dry in an aqueous medium on a commercial γ alumina support as in Example 1. The tungsten precursor salt is ammonium metatungstate (NH₄)₁₀W₁₂O₃₉, x H₂O, the nickel precursor salt is nickel nitrate Ni(NO₃)₂, 6 H₂O. The quantity of nickel is adjusted in order to keep to the molar ratio Ni/(Ni+W)=0.3. The catalyst is dried and calcined under the conditions described in Example 1. On this non-sulphurated catalyst Ni—WO₃/Al₂O₃, the tungsten content is 15.4%, which corresponds to 0.104 mole of the element tungsten per 100 g of catalyst, the nickel content is 2.0%, which corresponds to 0.045 mole of the element nickel per 100 g of catalyst. This catalyst B is representative of an industrial catalyst.

### Example 3

### Supported Ni—MoₓW₍₁₋ₓ₎ Catalysts (According to the Invention)

A series of catalysts containing at the same time molybdenum, tungsten and nickel is prepared. The molybdenum and tungsten are present in the form of a solid solution, at least in the sulphurated state, the atomic proportions of molybdenum and tungsten vary according to x, 0<x<1. The quantity of nickel is adjusted in order to keep the molar ratio Ni/(Ni+Mo+W)=0.3. These catalysts are prepared by co-impregnation of the molybdenum, tungsten and nickel precursor salts described in Examples 1 and 2, in aqueous medium, on the alumina described in Example 1. The damp solid obtained is left to rest under a humid atmosphere at a temperature of approximately 20° C., dried at a temperature of 120° C., then calcined at 500° C.

The molybdenum, tungsten and nickel contents on the non-sulphurated catalysts are given in Table 1. The total number of impregnated metal moles (molybdenum or tungsten) is always the same.

TABLE I
Composition of the different Ni-MoₓW₍₁₋ₓ₎ oxide catalysts containing
molybdenum and tungsten in the form of a solid solution after sulphuration

| Reference | | Mo | W | n(Mo) | n(W) | n₁ = n_{Mo} + n_{W} | Ni | n(Ni) |
|---|---|---|---|---|---|---|---|---|
| Catalyst | x | (wt. %) | (wt. %) | (mol · g⁻¹) | (mol · g⁻¹) | (mol · g⁻¹) | (wt. %) | (mol · g⁻¹) |
| C1 | 0.25 | 2.2 | 11.6 | 0.026 | 0.078 | 0.104 | 2.0 | 0.045 |
| C2 | 0.50 | 4.3 | 7.7 | 0.052 | 0.052 | 0.104 | 2.1 | 0.045 |
| C3 | 0.75 | 6.4 | 3.8 | 0.078 | 0.026 | 0.104 | 2.1 | 0.045 |

### Example 4

### Supported Ni—MoₓW₍₁₋ₓ₎ Catalysts being Presented in the Form of a Mechanical Mixture (Not According to the Invention)

A proportion x of the catalyst Ni—MoO₃/Al₂O₃ prepared according to Example 1 as well as a proportion (1-x) of the catalyst Ni—WO₃/Al₂O₃ prepared according to Example 2 are mechanically mixed. A series of catalysts of Ni—MoₓW₍₁₋ₓ₎ compositions given in Table II is thus prepared.

TABLE II
Composition of the different Ni-MoₓW₍₁₋ₓ₎ oxide catalysts containing
molybdenum and tungsten (not according to the invention)

| Reference | | Mo | W | n(Mo) | n(W) | n₁ = n_{Mo} + n_{W} | Ni | n(Ni) |
|---|---|---|---|---|---|---|---|---|
| Catalyst | x | (wt. %) | (wt. %) | (mol · g⁻¹) | (mol · g⁻¹) | (mol · g⁻¹) | (wt. %) | (mol · g⁻¹) |
| D1 | 0.25 | 2.2 | 11.6 | 0.026 | 0.078 | 0.104 | 2.0 | 0.045 |
| D2 | 0.50 | 4.3 | 7.7 | 0.052 | 0.052 | 0.104 | 2.1 | 0.045 |
| D3 | 0.75 | 6.4 | 3.8 | 0.078 | 0.026 | 0.104 | 2.1 | 0.045 |

### Example 5

### Sulphuration of the Oxide Catalysts Prepared Previously

The oxide catalysts A, B, C1 to C3, D1 to D3 are loaded into a fixed-bed reactor and sulphurated under a flow of H₂/H₂S (10 molar % of H₂S), at atmospheric pressure, at 450° C., for 2 hours.

### Example 6

### Analysis of the Supported Catalysts by X-Ray Absorption

The sulphurated catalysts according to Example 5 are analyzed by EXAFS. The analysis is carried out at the K threshold of molybdenum, between 19800 and 21000 eV, and at the L_{III} threshold of tungsten, between 10100 and 11000 eV, using synchrotron radiation, by measuring the intensity absorbed by a sample. The Fourier transforms obtained at the K threshold of molybdenum for catalysts A, C1, C2, C3 are given in FIG. 1. The Fourier transforms obtained at the L_{III} threshold of tungsten for catalysts B and C2 are given in FIG. 2. The Fourier transforms obtained at the K threshold of molybdenum for catalysts A, C2 and C3 are given in FIG. 3.

The number of molybdenum and tungsten atoms situated around an absorbing molybdenum atom is given in Table III. The number of molybdenum and tungsten atoms situated around an absorbing tungsten atom is given in Table IV.

TABLE III
Environment of an absorbing molybdenum atom

| Catalyst | N(Mo) | N(W) |
|---|---|---|
| A | 5.2 | 0 |
| C3 | 4.0 | 1.7 |
| C2 | 2.9 | 1.9 |
| C1 | 2.0 | 4.3 |
| D2 | 5.3 | 0 |

TABLE IV
Environment of an absorbing tungsten atom

| Catalyst | N(Mo) | N(W) |
|---|---|---|
| B | 0 | 4.3 |
| C2 | 2.0 | 2.6 |
| D2 | 0 | 4.4 |

EXAFS makes it possible to detect the presence of both molybdenum and tungsten within the same flake, around a given absorbing atom, for the series of catalysts C1, C2, C3 according to the invention. When the solid solution is formed, a molybdenum atom is surrounded with both molybdenum atoms and tungsten atoms (Table III) and a tungsten atom will be surrounded with both tungsten atoms and molybdenum atoms (Table IV). It is in fact observed (FIG. 1) that the intensity of the second peak, situated around 2.8 Å, reduces and a third peak appears, around 3.2 Å, for catalyst C1. Quantitatively, Table III shows that the number of molybdenum neighbours reduces and the number of tungsten neighbours increases when the proportion x of molybdenum reduces in the solid solution. Table IV shows that the W neighbours appear (catalyst C2). On the other hand, in the case of a mechanical mixture of Ni—MoS₂/Al₂O₃ and Ni—WS₂/Al₂O₃ a molybdenum atom is surrounded only by molybdenum atoms (5.2 atoms, Table III, catalyst D2) and a tungsten atom is surrounded only by tungsten atoms (4.3 atoms, Table IV, catalyst D2).

The differences between the catalysts according to the invention (C2 for example) and the catalysts not according to the invention (D2 for example) are clearly illustrated by FIG. 3 and Tables III and IV.

### Example 7

### Tetraline Hydrogenation Test (Aromatic Model Molecule)

The activity of the catalysts supported on alumina A, B, C1 to C3, D1 to D3 previously prepared and sulphurated was compared in aromatic molecule hydrogenation (hydrogenation of tetraline), in a fluidized fixed bed, under hydrogen pressure, under the following operating conditions:
Total pressure: 45 bar
Tetraline pressure: 6000 Pa
H₂S pressure: 1504 Pa
Temperature: 300° C.
Hydrogen flow rate: 300 ml.min⁻¹
Mass of catalyst charged: 87 mg

The catalytic performances are given in Table V. They are expressed as a gain in activity relative to the activity (in moles of tetraline converted per g of catalyst and per second) of the weighted average of the activities of the reference catalysts A and B, or in activity relative to the activity of catalyst A.

TABLE V
Activity of the supported catalysts in the hydrogenation of tetraline

| Catalyst | Gain in activity | Activity relative to A |
|---|---|---|
| B | / | 1.22 |
| C1 | 13% | 1.32 |
| C2 | 33% | 1.45 |
| C3 | 65% | 1.75 |
| D1 | 0% | 1.16 |
| D2 | 0% | 1.11 |
| D3 | 0% | 1.05 |
| A | / | 1 |

The catalysts containing both molybdenum and tungsten forming the solid solution (C1 to C3) have an activity greater than the weighted average of the activities of the reference catalysts A and B. Moreover, these catalysts are more active than catalyst A only containing molybdenum or catalyst B only containing tungsten. The activities of catalysts D1 to D3 correspond perfectly to the weighted averages of the activities of catalysts A and B.

### Example 8

### HDS Test of a Gasoil

The catalytic activity of catalyst C2 according to the invention was studied in the HDS of a gasoil with 146 ppm of sulphur, in a fluidized fixed bed. The experimental conditions are given in Table VI.

TABLE VI
Gasoil test conditions

| | |
|---|---|
| Sulphur content of the gasoil | 146 ppm |
| Reaction temperature | 340° C. |
| H₂ pressure | 3 MPa |
| HSV | 4 h⁻¹ |
| H₂S pressure | 0-0.09 MPa |

The catalytic performances are given in Table VII. They are expressed as a gain in activity relative to the weighted average of the activities of the reference catalysts A and B, or in activity relative to the activity of catalyst A.

TABLE VII
Activity of the supported catalysts in the HDS of a gasoil

| Catalyst | Gain in activity | Activity relative to A |
|---|---|---|
| A | / | 1 |
| C2 | 35% | 1.25 |
| B | / | 0.8 |

With catalyst C2, the gain in activity is 35% relative to the catalytic activity of a mechanical mixture, of the same overall composition, of catalysts A and B. This example makes it possible to show that the gain in activity observed on a model molecule is retained on an actual charge.

### Example 9

### Sulphuration According to the Invention

The oxide catalysts A, B, C1 to C3, D1 to D3 are loaded into a fixed-bed reactor and sulphurated ex-situ under a flow of H₂/H₂S (15 molar % of H₂S) at atmospheric pressure, at 450° C., for 2 hours. They are then transferred into the fixed-bed reactor of the toluene hydrogenation test.

### Example 10

### Sulphuration Not According to the Invention

The oxide catalysts A, B, C1 to C3 are loaded into the fixed-bed reactor of the toluene hydrogenation test and sulphurated in-situ under a mixture of hydrocarbon and sulphurizing agent, at 330° C., for 2 hours.

### Example 11

### Hydrogenation of Toluene—Catalytic Activity of Catalysts A, B, C1 to C2 After Sulphuration According to the Invention or Not According to the Invention

The catalytic activity of catalysts A, B, C1 to C2 after sulphuration according to the invention or not according to the invention was studied in the hydrogenation of toluene in a fluidized fixed-bed reactor. The experimental conditions are given in Table VIII

TABLE VIII
Toluene test conditions

| Reaction temperature | 330° C. |
|---|---|
| Total pressure | 60 bar |
| HSV | 2 |

The charge is composed in wt. % of 20% of toluene, 74.6% of cyclohexane, 5.4% of DMDS.

The catalytic performances are given in Table IX. They are expressed as a gain in activity relative to the weighted average of the activities of the reference catalysts A and B.

TABLE IX
Gain in activity of the supported catalysts in the hydrogenation
of toluene after sulphuration according to the invention or
not according to the invention

| | Sulphuration according to | Sulphuration not according |
|---|---|---|
| | the invention | to the invention |
| Catalyst | Gain in activity | Gain in activity |
| A | / | / |
| C3 | 15% | 5% |
| C2 | 35% | 0 |
| C1 | 50% | −10% |
| B | / | / |

Catalysts C1 to C3 containing both molybdenum and tungsten within the same solid solution and having undergone sulphuration according to the invention which allows the formation of the solid solution, have a gain in activity relative to the weighted average of the activities of catalysts A and B of 15 to 50% according to the compositions in Mo and W.

Catalysts C1 to C3 containing both molybdenum and tungsten and having undergone sulphuration not according to the invention which does not allow the formation of the solid solution, do not have any significant gain in activity relative to the weighted average of the activities of catalysts A and B.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 03/11.032, filed Sep. 19, 2003 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Catalyst for the hydrorefining and/or hydroconversion of hydrocarbon charges characterized in that the active phase of said catalyst comprises at least one molybdenum and tungsten sulphide solid solution within the same flake, of approximate general formula MoₓW₁₋ₓS_{y}, where x is a number strictly comprised between 0 and 1 and y is a number comprised between 1.4 and 2.6 and in that the catalyst is subjected to a sulphuration treatment consisting of bringing the catalyst into contact ex situ with an H₂/H₂S flow at atmospheric pressure, at a temperature comprised between 350 and 600° C.

2. Catalyst according to claim 1, which comprises at least one element of Group VIII.

3. Catalyst according to claim 2, such that said element of Group VIII is chosen from the group formed by iron, cobalt and nickel.

4. Catalyst according to claim 1, containing at least one doping element chosen from the group formed by boron, phosphorus, silicon and the halogens.

5. Catalyst according to claim 1, which comprises a matrix.

6. Catalyst according to claim 5 in which the matrix is chosen from the group formed by alumina, silica, silica-alumina, magnesia, clay, titanium oxide, zirconium oxide, lanthanum oxide, cerium oxide, aluminium phosphates, boron phosphates, alumina boron-oxide, alumina titanium-oxide, alumina-zirconia and titanium-oxide zirconia combinations, magnesium, calcium, barium, manganese, iron, cobalt, nickel, copper and zinc aluminates, and (zinc, nickel, cobalt) titanates alone or in mixture.

7. Catalyst according to claim 5 which contains at least one synthetic or natural simple clay, delaminated or not delaminated, of the 2:1 dioctehedral phyllosilicate or 3:1 trioctahedral phyllosilicate type, such as kaolinite, antigorite, chrysotile, montmorillonite, beidellite, vermiculite, talc, hectorite, saponite, laponite.

8. Catalyst according to claim 1 which contains at least one compound chosen from the group formed by the family of non-zeolite molecular sieves such as the mesoporous silicas, silicalite, silicoaluminophosphates, aluminophosphates, ferrosilicates, titanium silicoaluminates, borosilicates, chromosilicates and aluminophosphates of transition metals (including cobalt).

9. Mass catalyst according to claim 1 containing in wt. % relative to the total mass of the catalyst:
0.01 to 100% of at least one molybdenum and tungsten sulphide solid solution phase, the catalyst being able to contain moreover
0 to 99.95% of at least one element of Group VIII,
0 to 20% of at least one doping element

10. Supported catalyst according to claim 1 containing in wt. %, relative to the total mass of the catalyst
0.1 to 99% of at least one molybdenum and tungsten sulphide solid solution phase,
1 to 99.9% of at least one matrix,
the catalyst being able to contain moreover
0.30% of at least one metal of Group VIII,
0.90% of at least one zeolite molecular sieve
0 to 20% of at least one doping element.

11. Catalyst according to claim 10 where the zeolite molecular sieve is chosen from the group formed by Y zeolite, fluorinated Y zeolite, Y zeolite containing rare earths, X zeolite, L zeolite, beta zeolite, mordenite with small pores, mordenite with large pores, omega zeolites, Nu-10, ZSM-22, Nu-86, Nu-87, Nu-88 and the ZSM-5 zeolite.

12. Catalyst according to claim 11 in which the zeolite molecular sieve is a Y zeolite with a faujasite structure.

13. Hydrorefining and/or hydroconversion process for hydrocarbon charges using the catalyst according to one of the preceding claims, at a temperature above 200° C., a pressure comprised between 1 MPa and 30 MPa, in the presence of hydrogen with a hydrogen/hydrocarbons H₂/HC volume ratio comprised between 10 and 5000 litres of hydrogen per litre of charge and at an hourly space velocity comprised between 0.1 and 10^{h-1}.

14. Process according to claim 13 such that the charge used in the process is chosen from the group formed by gasolines, gasoils, vacuum gasoils, deasphalted or non-deasphalted residues, paraffinic oils, waxes and paraffins and effluents from the Fischer-Tropsch process.
